(19) United States
(12) Patent Application Publication
(10) Pub. No.: US 2018/0326163 A1
(43) Pub. Date: Nov. 15, 2018

(54) ATTACHMENT FOR AN INJECTION PEN OR SYRINGE

(71) Applicant: TICKLETEC LTD, West Sussex (GB)

(72) Inventor: Ralph-Peter Steven BAILEY, West Sussex (GB)

(21) Appl. No.: 15/775,886

(22) PCT Filed: Nov. 18, 2016
(86) PCT No.: PCT/EP2016/025149

§ 371 Date: May 14, 2018

(30) Foreign Application Priority Data
Nov. 18, 2015 (GB) 1520305.2
Aug. 1, 2016 (GB) 1613260.7

(51) Int. Cl. A61M 5/42; A61M 5/32

(52) U.S. Cl. CPC A61M 5/425; A61M 5/3287

(57) ABSTRACT

An attachment for an injection pen or syringe, the attachment being attachable to the needle or needle end of an injector pen (7) or syringe, the attachment comprising a circumferential array of inwardly pointing flexible fingers (9) configured such that when the flexible finger tips are pressed against the injection site the flexible fingers are capable of flexing axially and inwardly to promote the gathering of a pucker of subcutaneous tissue in the vicinity of a needle insertion point.

### FIELD

This invention relates to an attachment for an injection pen or syringe. In preferred embodiments the devices disclosed herein can improve the safety, comfort and efficacy of the self-administration of an injectable drug, for example of insulin.

### BACKGROUND

A significant user group who need to inject themselves are diabetics. Diabetics have a need to regularly inject insulin which being a lipid and a protein would be digested if taken orally and otherwise be attacked by the immune system in the gastrointestinal tract. Type 1 diabetics can only treat their condition by injection, as with a significant proportion of Type 2 sufferers.

Whilst self-injection is an effective way to manage the condition, it is fraught with risk factors that can compromise its effectiveness.

When injecting it is important for the needle to penetrate the epidermis (which is ˜2 mm thick) so that it can deliver insulin (in this particular example) into the fatty subcutaneous tissue that has a thickness which can vary by several millimetres. From there it can infuse at a controlled rate into the bloodstream. If the needle is not inserted deep enough into the skin of the user it can blister the epidermis and it will take longer for the insulin to be absorbed, often resulting in hyperglycaemia. If too deep the needle can enter the muscle tissue where the insulin would be absorbed too quickly, resulting in a rapid drop in blood sugar and a potentially dangerous hypoglycaemic episode. Because the preferred tissue is inherently soft and injector ‘pens’ don't have a large surface area end stop the resulting needle depth is significantly proportional to application pressure.

There is a technique that improves the chance of injecting successfully into the appropriate layer of subcutaneous tissue. It is called the “pinch” where the skin and underlying tissue is puckered between finger and thumb, thereby locally increasing the depth of the fatty region where the insulin is best deposited. This reduces the risk of under or over depth penetration. The problem is that it requires two hands, and the best injection sites like in the kidney area are not easily accessible in this way. The pinch does though have a further beneficial effect. It is known that creating a sensory input at a pain site acts as a nerve distraction that mitigates the discomfort. That is why it is instinctive to rub the site of a painful trauma.

Another drawback associated with conventional self-injection equipment, particularly when dealing with child patients, is that the visible needle of the syringe or pen can cause significant distress to those patients who are needle-phobic.

One previously proposed injection pen is disclosed in United States patent publication number US2011/0166509. This document discloses a number of different injectors, one of which (shown in FIGS. 3a to 4d) replicates the aforementioned pinch technique by providing fingers that can be manipulated by attached handles to create a pinch of skin that a medicament can then be injected into. A problem with this arrangement, however, is that the device still requires two hands to operate (one to pinch the skin and one to operate the syringe) thereby rendering it unsuitable for injection into less accessible areas of a patient's body. A second injector (shown in FIGS. 5a to 5d) provides a pair of helical fingers that twist when pressed against the user's skin to lift a section of skin prior to injection. Whilst this arrangement should provide for one-handed operation, the torsional forces caused by twisting the fingers against the skin can make it difficult to stabilise the injector during the injection process, thereby raising the possibility of the injection needle inadvertently sheering through the tissue laterally and causing an injury.

Aspects of the present invention have been devised with the aforementioned problems in mind. In one aspect the present invention has been devised to facilitate the injection process, particularly but not exclusively to improve the efficacy of such injections.

### SUMMARY

A presently preferred embodiment of the present invention provides an attachment that comprises a plurality of fingers which are capable of moving radially inwardly towards one another to gather a pucker of skin tissue between the fingers as an injection pen or syringe to which the attachment is fitted is used to inject a patient (in the preferred arrangement, via a needle insertion point within the pucker). In one preferred embodiment of the invention, the fingers are attached to pads that gather the pucker of skin. In another embodiment the pucker of skin is gathered directly between the fingers themselves.

One implementation of the teachings of the present invention provides an attachment for an injection pen or syringe, the attachment comprising a generally annular support that is fittable to the needle or needle end of an injector pen or syringe, and a circumferential array of fingers coupled to said annular support so that said fingers extend axially from said pen or syringe when said attachment is coupled thereto, said fingers being configured to move radially inwardly in response to an axial force applied to the pen or syringe as the injection pen or syringe is used, the radial inward movement of said fingers causing a pucker of tissue to be gathered between said fingers in the vicinity of a needle insertion point.

A second implementation of the teachings of the invention provides an attachment for an injection pen or syringe, the attachment being attachable to the needle or needle end of an injector pen or syringe, the attachment comprising a circumferential array of inwardly pointing flexible fingers configured such that when the flexible finger tips are pressed against the injection site the flexible fingers are capable of flexing axially and inwardly to promote the gathering of a pucker of subcutaneous tissue in the vicinity of a needle insertion point.

A preferred embodiment of the invention can be attached to a single use needle after it has been attached to a pen type injector or epi-pen.

In one embodiment the device is comprised of two plastic parts, a stiff injection moulding that is designed to clip onto a universal standard fit needle and an elastomeric part that attaches to the moulding and features a radial array of protruding ‘fingers’.

These ‘fingers’ are designed to fold inwards when their tips are pressed against the users skin. Their sections thin out dramatically becoming functionally a hinge line close to where the fingers join together at a common band. The band then stretches over a lip on the plastic holder which thereby retains it. This hinge line subsequently guides and facilitates the folding back of the thicker and hence stiffer fingers. As they fold about this pivot axis they also displace inwards towards the needle. In this embodiment the outer facing side of the finger is curved around a notional focal point giving it the aspect of a segment of a sphere. It is then covered on its outwards facing side with tooth like texture features designed to grip the skin as the finger tilts inwards.

As the motion progresses the finger tips drag the underlying skin and subcutaneous layer with them in a manner akin to the pinch process. The action is in fact superior, as the pinch is not just single axis between a finger and thumb but acts between the radial array of fingers, which in one embodiment number twelve. The resulting pucker is then highly suitable for injecting insulin into it as it comprises of a pile of the ideal subcutaneous tissue. Consequently needle placement is more tolerant of variation in penetration depth and better able to defuse and distribute the insulin infusion.

The greater surface area presented by the array of inward folded fingers compared with the end of a basic injector, acts like a pad that prevents the needle penetrating too deeply even if excess pressure is applied. The folded fingers with their texture features also grip the skin preventing it sliding sideways if the pen position is insufficiently controlled, or at least if there is any sideways motion the fingers take the patch of skin adjacent to the needle with them, thereby avoiding the needle sheering through the tissue laterally, which could cause an injury.

The pinch action stimulates and saturates the local nerves, distracting and obscuring the discomfort caused by inserting the needle. It is like scratching an itch or rubbing an ache. Furthermore as the needle is unlikely to penetrate beyond the subcutaneous tissue it is less likely to hit a nerve in the underlying muscle.

The injector pen piston can then be depressed as usual, introducing the insulin. After the needle has subsequently been withdrawn the fingers tend to spring outwards, dragging and stretching the skin between them. This helps to seal the needle puncture point through which the injected insulin might otherwise leak.

The open array of fingers tends to obscure the needle and as such gives comfort to those who may suffer from needle phobia, especially the image of the needle penetrating the skin.

In a preferred arrangement, the attachment may be attached to the needle shell after it has been screwed onto the insulin pen. At this stage the safety cap for the needle tip is still in place. The flexible nature of the fingers enables the user to push them aside in order to grip the cap protecting the needle (where fitted) and pull it off. Similarly once the injection has been given the cap can be replaced to protect the needle before the device is removed from the needle shell. This process mitigates the risk of sharps injury.

In a preferred implementation, the attachment is designed specifically to clip onto a single use universal fit needle as used by pen-like injectors typically employed for injecting insulin. However an alternative fitting for the elastomeric component would enable the key features of distraction analgesia and needle obscuring to be enjoyed by other syringe applications that might otherwise give distress, anxiety or discomfort to the patient—particularly when applied to children.

In such an embodiment the holder features a pad that can be held against the skin by a user's two fingers and, optionally, a conical shield that guides the needle through a hole in the pad. Such a shield could be hyperbolic, curving open at an accelerating rate so as to provide protection for the user's fingers that will otherwise be holding the pad down and so mitigates the risk of sharps injury.

A smaller version of the elastomeric pad could be envisaged designed specifically for dental anaesthetic injections. It creates a more effective pinch than a dentist could generate with his fingers by virtue of its proliferation of texture points. In one embodiment the pad holding the flexing component may be held from one side by a handle that enables the pads angle to the handle to be adjusted, as dictated by the imperatives of access into difficult to reach places. In another embodiment the head may be pivoted such that it will self-align to any surface against which it is pushed.

A third implementation of the teachings of the invention provides an attachment that is attachable to the needle or needle end of an insulin injector pen that can open an array of ‘fingers’ both axially and radially about a needle such that the axial force caused by subsequently bearing the finger's tips against the injection site causes them to retract back and together in a path that is the reverse of their deployment thereby gathering a pucker of tissue emulating the manual ‘pinch’ technique that concentrates the subcutaneous fat layer under the tip of the needle.

In this aspect of the invention the attachment may be attached to the needle end of a pen type injector after the disposable needle has been fitted as usual.

The ‘fingers’ may be extended by stored mechanical energy in preferably an outward and separating motion. The finger's tips may advantageously be covered in a soft high friction elastomer and/or have a surface topography that promotes good adhesion to any contacting skin. Such topography may beneficially be comprised of a radial array of features that enable surface hair to fall between the peaks and thereby not prevent good frictional adhesion.

In one embodiment the three fingers are steered outwards by running in tracks on both the fingers and the device body such that as they swing out they also tilt their tips apart thereby extending their reach. When the user then presses the device against their skin, the force on the fingertips causes them to reverse their opening path, retracting back together into their closed position. As the motion progresses the tips drag the underlying skin and subcutaneous layer with them in a manner akin to the pinch process. The action is in fact superior, as the pinch is not just single axis between finger and thumb but two axis between three (or more) fingertip pads. The resulting pucker is then highly suitable for injecting insulin (for example) into it as it provides a larger volume of suitable tissue, consequently more tolerant of variation in needle depth and better able to distribute the insulin infusion.

The greater surface area presented by the pad array compared with the end of a basic injector, combined with the increased resilience of the stretched tissue around the pucker reduces the denting of the soft tissue that could result in the needle penetrating too deeply. The pads also support the position of the needle where it might otherwise be difficult to hold it steady, reducing any damage that might be caused by the needle shearing through the tissue laterally.

It is also the case that the pinch action tends to stimulate and distract the local nerves, obscuring the discomfort caused by inserting the needle. Furthermore, as the needle is unlikely to penetrate beyond the subcutaneous tissue it is less likely to hit a nerve in the underlying muscle.

In this implementation, the injector pen piston can then be depressed as usual, introducing the insulin. After the needle has subsequently been withdrawn the finger pads are once again pushed outwards by the action of the spring, dragging and stretching the skin between them. This helps to seal the needle puncture point through which the injected insulin might otherwise leak.

The device may include a number of passive moveable markers or a dial which can be set by the user as an aide-memoire as to when the injector was last used.

In one embodiment the device may include a processor that reports when it is used by means of a wireless communications link (for example, a WiFi or Bluetooth link) to an app running (for example, in background mode) on a smartphone of the user. Such an app can refer to a record of a preferred injection regime in order to decide whether to implement an alert to the user if no usage is reported.

Another implementation of the teachings of the invention (depicted schematically in FIGS. 5 to 7) provides apparatus for use with an injection pin or syringe, the apparatus comprising a generally annular support, and a circumferential array of fingers coupled to said annular support so that said fingers extend axially from one side of said support, at least part of said fingers being configured to move radially inwardly in response to an axial force applied to the support and thereby gather a pucker of tissue between said fingers in the vicinity of a needle insertion point. In one embodiment a second side of said support may include a guide for a needle of an injection pen or syringe. The guide may be conical. In another embodiment the support may be moveably coupled (for example by means of a gimbal) to a handle. In this arrangement the support self-aligns to a skin surface against which the fingers bear.

A common advantage of arrangements disclosed herein, is that as the size and complexity of the component necessary to achieve a bespoke fitting is reduced, the attachment can be readily exchanged as required to suit each type of injector pen. In another implementation the attachment could be fitted to the replaceable needle rather than the pen itself. An advantage of this implementation, is that the attachment would be more universal as the range of alternate needle designs is much smaller than the range of pens.

Advantageously, the arrangements disclosed herein can provide a means to better control needle depth whilst self-injecting. Another advantage associated with these arrangements is that they can create a pucker to promote the needle tip entering the desirable layer of subcutaneous tissue—thereby enabling injections to be safely given at awkward body sites where it is difficult to apply sufficient physical control of steady positioning and pressure. A further advantage of the arrangements disclosed herein is that they can mitigate the discomfort associated with the act of injection. Other advantages associated with the arrangements disclosed herein are: (i) that they can fit most styles of injector pen; (ii) that they can provide distraction analgesia for any hypodermic injection, and (iii) that by at least partially obscuring the needle they can be useful for needle-phobic patients.

Although the following detailed description refers to injections in the arm or other body part of a patient, it will be appreciated that this is merely illustrative and not intended to be a limitation of the scope of the present invention. For example, it will be appreciated that a variant of the device could be used to mitigate the discomfort of oral injections. The device may also have veterinary applications.

### BRIEF DESCRIPTION OF THE DRAWINGS

Various presently preferred implementations of the teachings of the present invention will now be described, only by way of illustrative example, with reference to the accompanying drawings, in which:

FIG. 1 is a diagrammatic cross-sectional view of a portion of a user's skin illustrating the aforementioned pinch technique;

FIG. 2 is a schematic perspective view of an attachment according to a first embodiment of the invention axially aligned with an illustrative injector pen;

FIG. 3 is a schematic side elevation of an attachment and an illustrative injector pen, with the attachment depicted in various stages of operation;

FIG. 4 is a schematic perspective view of one of the fingers of the attachment shown in FIGS. 1 to 3;

FIGS. 5 and 6 are front and rear perspective views, respectively, of apparatus for facilitating the gathering of a pucker of skin;

FIG. 7 is a perspective view of apparatus embodying the teachings of the invention configured for use in dental applications;

FIG. 8 is a schematic view, partly in section, of an attachment according to a second embodiment of the present invention;

FIG. 9 is a schematic view, partly in section, of the attachment depicted in FIG. 8 in a retracted state;

FIGS. 10 and 11 are schematic depictions of the attachments shown in FIGS. 8 and 9, respectively, attached to an illustrative injector pen; and

FIG. 12 is an enlarged view of part of the attachment and injector pen depicted in FIG. 11.

### DETAILED DESCRIPTION

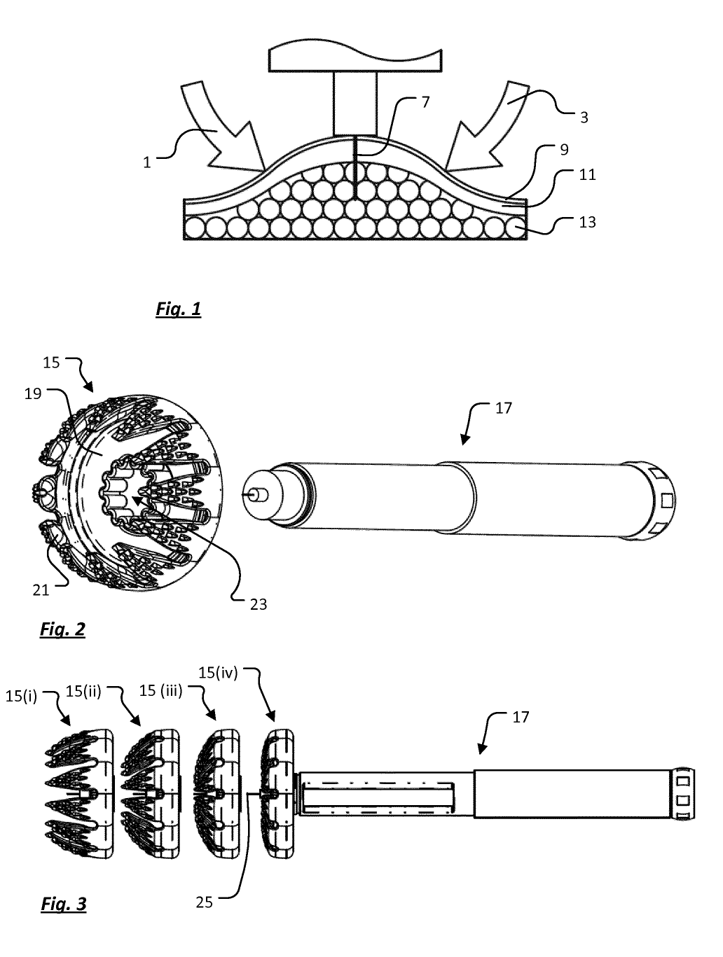

Referring now to FIG. 1 of the drawings, there is provided a diagrammatic representation, in cross-section, of a part of a user's skin showing the aforementioned “pinch” technique. As shown, by applying a force to the skin in directions 1 and 3, a user can raise a pucker of skin 5 into which a needle 7 can be inserted so that the needle extends through an epidermal skin layer 9, a dermis skin layer 11 and into subcutaneous tissue 13 that the needle should beneficially enter. As is well known in the art, by using the pinch technique it is less likely that a user will inadvertently insert the needle through the subcutaneous tissue and into the underlying muscle. Similarly, it is also less likely that the user will inadvertently insert the needle at an angle into the dermal skin layer.

FIG. 2 is a schematic perspective view of an attachment 15 according to a first embodiment of the invention axially aligned with an illustrative injector pen 17. The attachment 15 comprises a generally annular support 19 to which a circumferential array of fingers 21 is coupled. In this particular implementation of the teachings of the invention, the array comprises 12 fingers, but it will be appreciated that a greater or lesser number of fingers may be provided if desired.

The annular support includes an aperture 23 (preferably located generally centrally of the support so that the fingers are roughly equally spaced from the aperture) sized to enable (in this instance) the support to be fitted over the end of an injector pen 17. As will be appreciated, the aperture may be reduced in size in circumstances where the attachment is configured to be attached to the needle instead of the body of the pen. In preferred implementations the region of the support that defines the aperture 23 is convoluted so that the support can expand to grip the pen (in this instance) when the support is fitted thereto.

In one preferred implementation of the teachings of the invention, the support is of a plastics material and the array of fingers are formed of an elastomeric material (for example of silicone elastomer) that is stretched and fitted over the plastics support. Preferably the support is relatively stiff and the fingers are flexible. In one implementation, the fingers may be configured so that, once fitted to the support, the tips of the fingers project inwardly towards the aperture. By virtue of this arrangement, the fingers will tend to collapse towards the aperture 23 when the fingers abut a user's skin surface and the support is pushed towards the skin surface.

FIG. 3 is a schematic side elevation of an attachment 15(i) to 15(iv) and an illustrative injector pen 17, with the attachment depicted in various stages of operation. In the first illustration, the fingers of the attachment 15(i) are shown fully extended as they would appear before abutment against a user's skin. Attachments 15(ii) and 15(iii) show progressively increased collapse of the fingers due to the support being pushed against a user's skin. The attachment 15(iv) depicts the fingers in a fully collapsed position exposing a needle 25 attached to the injector pen 17. As will be appreciated, an advantage of this implementation is that the needle is substantially hidden from view until the fingers have significantly collapsed, which is useful when the attachment is used to inject users who are needle-phobic.

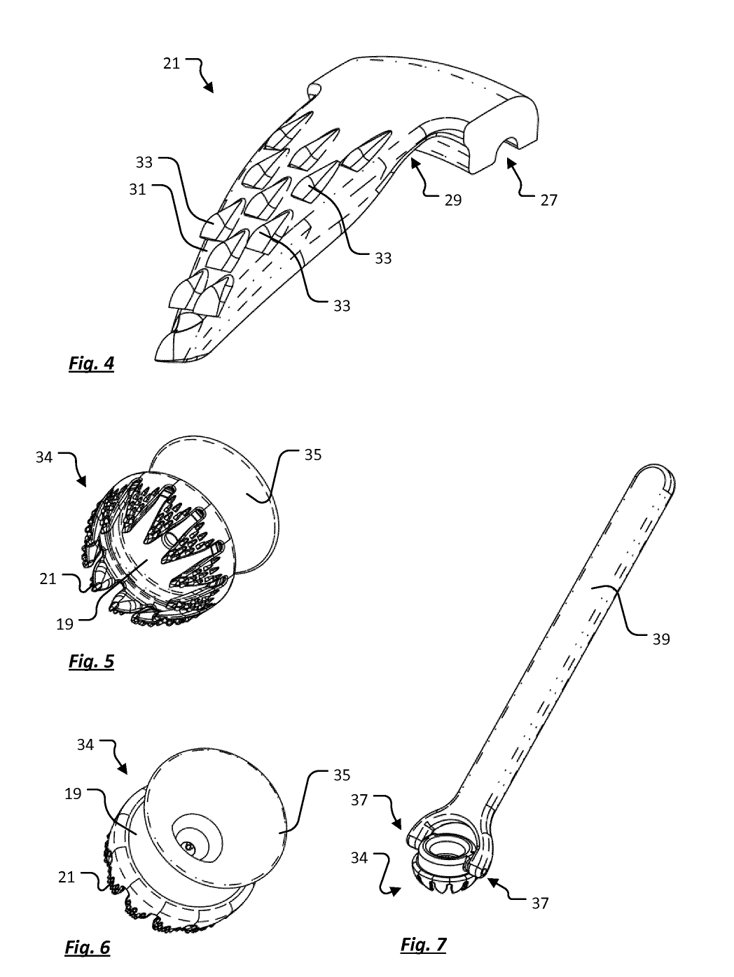

FIG. 4 is a schematic representation of one of the fingers 21 of the array. In FIG. 4 the finger 21 is shown separated from adjacent fingers for clarity, but it will be understood that normally the fingers will be joined together to form a ring that is fitted over the aforementioned support. To assist retention of the array on the support, the fingers of the array are provided with a groove 27 in which the support is retained when the array is fitted thereto. As shown, the fingers are locally thinned (relatively to adjacent portions) in a region 29 proximate that part of the array that fits over the support to from a hinge that causes the fingers to flex towards the support as the fingers of the attachment are pressed against the skin. A radially outward surface 31 of the fingers is provided with a plurality of skin-engaging formations 33 that are configured to grip the skin, as the attachment is pushed towards the skin, to drag the skin of the user up into to a pucker into which the needle of the injection pen (or syringe) is inserted. Additionally, the skin-engaging formations provide a sensory input that helps to distract the user from the sensation of the needle entering the skin.

FIGS. 5 and 6 are schematic representations of apparatus for use with an injection pen or syringe (but which does not necessarily couple to that pen or syringe). In this arrangement the support includes a guide 35 that extends from a side of the support opposite that from which the fingers extend. The aperture of the support is configured to enable a needle to pass through it, and the guide may be configured to be funnel shaped so that it protects the users fingers as the apparatus is pressed against the skin. FIG. 7 is a variant of the apparatus shown in FIGS. 5 and 6 that is particularly (but not exclusively) suitable for dental injections. In this implementation of the teachings of the invention, the apparatus 34 is coupled by a gimbal 37 to a handle 39 so that the apparatus can self-align when pressed against the gum tissue. In the arrangement depicted in FIG. 7, the guide has been omitted, although it may be beneficial for some applications to include a guide of the type depicted in FIGS. 5 and 6.

In the aforementioned implementations of the teachings of the invention the attachment may be considered to be akin to a flange with an elastomeric appendage that clips over the single use screw-on needle. This is a versatile attachment method as many pen designs exist, each with size and shape variations, but needle shapes are significantly standard—known as “universal fit”. To create a little radial preload to ensure reliable holding force the collar may be convoluted to facilitate a degree of band expansion.

The elastomeric component features a radial array of fingers attached to a common ring which may beneficially be moulded out of silicon, known for its inert nature and excellent shape recovery after flexing. To improve the friction and enhance the grip to the underlying skin an array of sharp edged protrusions may be included in the fingers outer surfaces, the features being aligned so as to optimize the friction and grip against the skin when the apparatus is pushed against the skin. The finger array ring component can simply stretch to clip over its holder or be bonded to it. It could be injection moulded out of a thermoplastic elastomer (TPE) to reduce its cost if produced in volume. It can also include an ionic silver additive to provide for antimicrobial properties.

Alternative material hardnesses can be employed for the flexing component. Hardnesses within the range of 50 Shore A and 90 Shore A appear to be particularly suitable. Different versions, with differing hardnesses, could be provided to meet the user's preference. Such hardness variations could be colour coded. This is analogous to users selecting their preferred hardness of toothbrush. The fingers could be provided with an anti-microbial additive to discourage fungal growth.

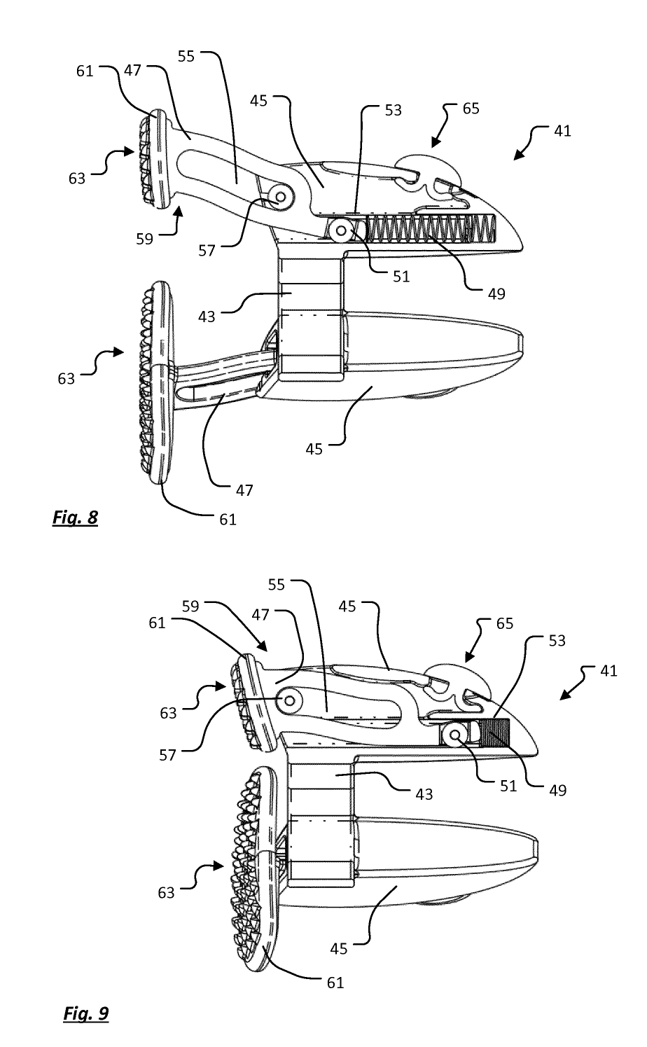

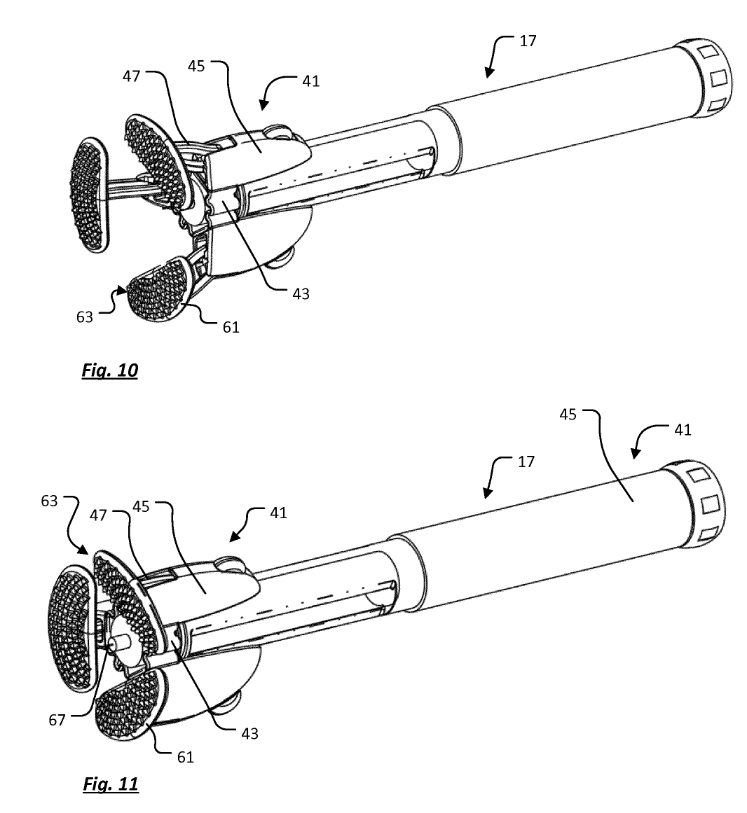

Referring now to FIGS. 8 and 9, there is depicted a schematic view, partly in section, of an attachment 41 according to another embodiment of the present invention.

In this implementation of the teachings of the invention, the attachment 41 comprises an annular support 43 to which three finger housings 45 (two of which are visible in FIGS. 8 and 9) are attached. One of the aforementioned finger housings 45 is shown in section. Each finger housing houses a finger 47 that is moveable into and out of the housing against a resilient bias 49 (in this particular example, a helical spring). FIG. 8 depicts the attachment with the fingers 47 fully extended from their respective housings 45, and FIG. 9 depicts the attachment with the fingers 47 fully withdrawn into their respective housings 45.

As with the aforementioned embodiments, the annular support 43 may include an aperture defined by a convoluted wall (visible in FIG. 12) so that the support can be expanded to grip on an injection pen or syringe push-fitted into the aperture.

Each of the fingers 47 is coupled to a roller 51 that is capable of moving up and down a channel 53 formed in the finger housing against the aforementioned resilient bias 49. Additionally, each finger includes a shaped slot 55 that guides a second roller 57 attached to the finger housing 45 as the finger moves into and out of the housing 45. The slot 55 is configured so that movement of the associated finger from the at-rest position shown in FIG. 8 towards the fully withdrawn position shown in FIG. 9 causes at least a tip portion 59 of the finger to move radially inwardly towards the aforementioned aperture in the annular support.

As shown in FIGS. 8 and 9, each finger includes an enlarged pad 61, and each pad includes a plurality of skin-engaging formations 63 on a side that abuts against a user's skin in use. As with previous embodiments, the skin-engaging formations 63 (which are preferably high-friction formations) function to drag the user's skin into a pucker as the attachment is pressed against a user's skin and the fingers move from the position shown in FIG. 8 towards the position shown in FIG. 9. Additionally, the skin-engaging formations provide sensory input that may serve as a distraction to the sensation of a needle entering the user's skin.

As will be appreciated by those persons of ordinary skill in the art, with the attachment coupled to an injection pen or syringe, pushing the syringe against a user's skin will cause the fingers to withdraw into their respective housings and create a skin pucker that a needle attached to the pen or syringe pierces. Once pierced, the medicament in the syringe or pen can then be injected into the user. Injection of the medicament may be accomplished after the pucker has been formed, or alternatively the creation of the pucker and the injection of medicament may be accomplished in a single step.

In this particular embodiment of the invention, at least one of the finger housings may include a status indicator 65 that is moveable from a first position shown in FIG. 8 to a second position shown in FIG. 9. The status indicator may be moved automatically as the attachment is operated to move the fingers into the housing, or alternatively may be manually set by the user once the attachment has been operated. In either case, the position of the status indicator serves as an aide memoire for the user as to whether or not the attachment has been operated, and hence whether or not they have used the attachment to inject their medicament.

In another envisaged implementation, the attachment may include a controller operable to determine when the attachment is used, and responsive to use to send a wireless signal to an associated processor—for example to send a signal (for example via Wi-Fi or Bluetooth™) to a smartphone running an application that records the times at which the attachment is used. The application may also be programmable with an injection timetable for a user, and be configured to remind a user in the event that they forget to inject themselves.

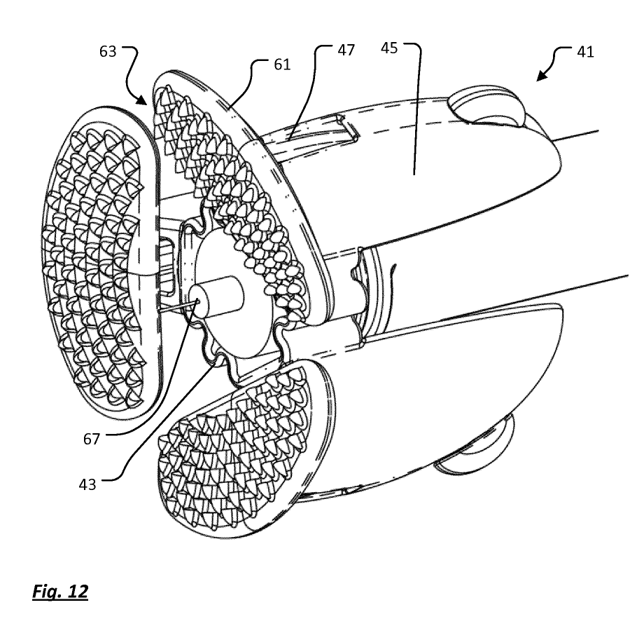

FIGS. 10 and 11 are schematic depictions of the attachments shown in FIGS. 8 and 9, respectively, attached to an illustrative injector pen 17. As described above, pushing the annular support 43 towards a user's skin causes the fingers 47 to retract (against the aforementioned resilient bias) into their respective housings 45. As the fingers retract a needle 67 attached to the injector pen is gradually exposed and driven into a pucker of tissue raised by the skin-engaging formations 63 on the pads 61

FIG. 12 is an enlarged view of part of the attachment and injector pen depicted in FIG. 11. As depicted, movement of the annular support 43 towards the user's skin has caused the fingers to draw towards one another (thereby causing a pucker in the user's skin) and has simultaneously exposed a needle 67 attached to the injector pen.

In a modification of this implementation of the invention the support may be configured to be clipped over the single use screw on needle of a syringe or injector pen. Such an arrangement may be more versatile as many pen designs exist, each with size and shape variations, but needle shapes are substantially standard.

The finger pads grip the skin and subsequently form the pucker. To improve friction an array of sharp edged protrusions may be moulded into an elastomeric cap, the features being aligned so as to optimize the friction when pulled inwards. The cap can clip over the pad, be bonded to it or be co-moulded onto it. The elastomer can include an ionic silver filler to provide for antimicrobial properties.

As will be appreciated from the foregoing, in this implementation of the invention the channel in the finger housing is axial, but in the finger the slot is curvilinear to create the desired motion profile. As the finger is ejected from the body by a compression spring, it is held between the two rollers that guide it out and up. The track profile favours it moving largely axial to start with but then increasingly outwards. This is the same path by which the fingers retract to create the pucker.

As well as being trapped by the rollers, the fingers also operate within a slot in the body that keeps them laterally stable. The rollers may be fabricated from short sections of PTFE tube with a steel pin axle. Alternatively instead of rollers they could be comprised of low friction bearing pads. A benefit of such a construction is that neither the fingers nor the support feature any undercuts. They can therefore be injection moulded out of simple two-part tooling.

Whilst several presently preferred embodiments have been described herein, it will be appreciated that these embodiments are merely illustrative and that modifications and alterations may be made thereto without departing from the spirit and scope of the present invention. For example, whilst reference is made herein to use with an injection pen, it will be appreciated that the attachment could equally be used with a conventional syringe. It should also be remembered that whilst certain presently preferred combinations of features have been set out in the accompanying claims, the scope of the present invention is not limited to these combinations but instead extends to include any combination or permutation of the features herein disclosed.

1. An attachment for an injection pen or syringe, the attachment being attachable to the needle or needle end of an injector pen or syringe, the attachment comprising a circumferential array of inwardly pointing flexible fingers configured such that when the flexible finger tips are pressed against the injection site the flexible fingers are capable of flexing axially and inwardly to promote the gathering of a pucker of subcutaneous tissue in the vicinity of a needle insertion point.

2. An attachment according to claim 1 wherein the fingers each have a base remote from their tip, and are thinned out in the vicinity of their base so as to create a preferred hinge line about which they can flex when axially loaded from their tips.

3. An attachment according to claim 1, wherein the fingers are attached to a common band which in turn is attached to a collar that can be fitted to the body of a single use needle or to the body of an injection pen or syringe.

4. An attachment according to claim 1, wherein the outward face of the fingers that contact the skin have features designed to enhance the grip between the fingers and the skin.

5. An attachment according to claim 1, wherein as the needle is withdrawn the fingers spring outward to stretch the skin and tissue between them thereby helping to seal the needle's puncture point.

6. An attachment according to claim 1, wherein the finger's tips have features that stand axially proud of the tip of the needle so as to contact the skin ahead of the needle such that they stimulate the local nerves on skin contact tending to saturate them with sensory input to mitigate and mask the discomfort that may be caused by the subsequent insertion of the needle.

7. An attachment according to claim 1, wherein the collapsed array of fingers together form a pad that limits the insertion depth of the needle by spreading the contact pressure over a large area.

8. An attachment according to claim 1 which is attachable to the body of the standard ‘universal’ size single use needle rather than directly to the pen.

9. An attachment according to claim 1, wherein the collar that fits over the needle body is convoluted so as to provide a degree of expansion to make it to help maintain a consistent grip and that is tolerant of poor manufacturing tolerances.

10. An injector comfort aid that has a radial array of flexible fingers which when pressed against the skin create a pinch effect against the skin which stimulates the local nerves tending to saturate them with sensory input to mitigate and mask the discomfort that may be caused by the subsequent insertion of a needle, optionally through the gap left in the centre of the array.

11. An aid according to claim 1 wherein the array of fingers is attached to a funnel like fitting designed to guide the insertion of a needle through the centre of the array and protect the users own fingers from possible sharps accidents which otherwise will be holding the fitting against the skin.

12. An aid according to claim 1, wherein the array of features designed to stimulate the local nerves is supported by a gimbal in turn attached to a handle that permits the feature array to self-align to the tissue against which it is being applied.

13. An attachment for a needle or a needle end of an insulin injector pen, wherein the device can open an array of ‘fingers’ both axially and radially about a needle such that the axial force caused by subsequently bearing the finger's tips against the injection site causes them to retract back and together in a path that is the reverse of their deployment thereby gathering a pucker of tissue emulating the manual ‘pinch’ technique that concentrates the subcutaneous fat layer under the tip of the needle.

14. An attachment as claimed in claim 1 where the path along which the ‘fingers’ retract is defined by sliding or olling along a track that can vary the proportion of radial to axial motion such that initially the motion to generate the pucker has a large radial component and as the needle enters the injection site the preferred motion becomes axial.

15. An attachment as claimed in claim 1, wherein as the needle is withdrawn the fingers are once again extended by a resilient bias to stretch the skin and tissue between them thereby helping to seal the needle's puncture point.

16. An attachment as claimed in claim 1 which includes marker elements that can be displaced into different positions (optionally, such positions can indicate the time of day when the attachment it was last employed and as such can act as an aid memoire as to previous usage).

17. An attachment as claimed in claim 1 where the fingers are retained by two rollers or slides, one on the device body that is trapped to move along a track on the finger and the other on the end of the finger that is trapped to move along a track on the body, with a spring means to provide for forward motive force to extend the fingers.

18. An attachment as claimed in claim 1, which is substantially attached to the body of the standard size single use needle rather than directly to the various sized pen.

19. An attachment according to claim 1 comprising a controller that is operable to register whether the device has been used and can report by wireless means to a smartphone or similar able to signal any failure to inject on schedule to the user.

20. An attachment for an injection pen or syringe, the attachment comprising a generally annular support that is fittable to the needle or needle end of an injector pen or syringe, and a circumferential array of fingers coupled to said annular support so that said fingers extend axially from said pen or syringe when said attachment is coupled thereto, said fingers being configured to move radially inwardly in response to an axial force applied to the support as the support is moved towards a user's skin, the radial inward movement of said fingers causing a pucker of tissue to be gathered between said fingers in the vicinity of a needle insertion point.
